(19) United States
(12) Patent Application Publication
(10) Pub. No.: US 2011/0118586 A1
(43) Pub. Date: May 19, 2011

(54) MEDICAL IMAGING APPARATUS CONTROLLER AND METHOD THAT ACCOMMODATE DIFFERENT CONTROL PROGRAM VERSIONS

(76) Inventor: Swen Campagna, Engelthal (DE)

(21) Appl. No.: 12/949,195

(22) Filed: Nov. 18, 2010

(30) Foreign Application Priority Data
Nov. 18, 2009 (DE) 10 2009 053 743.0

(51) Int. Cl. A61B 5/055

(52) U.S. Cl. 600/410

(57) ABSTRACT

In a method, a system and a computer program product to control a medical imaging apparatus or to extend a control method for such an apparatus, the divided into different segments. A physical segment includes all control modules that are dependent on the platform of the medical apparatus. A logical segment includes all control modules that are independent of a platform. The logical segment of the control method is swapped out to an additionally provided auxiliary control computer while the physical segment remains on the (previous) control computer. At the previous control computer a data transfer module is provided that receives data of the auxiliary control computer and relays it to the medical apparatus for control. The auxiliary control computer advantageously possesses standardized interfaces.

### BACKGROUND OF THE INVENTION

1. Field of the Invention

The present invention concerns a method, a system and a storage medium to control medical apparatuses (for example a magnetic resonance apparatus) by means of a control method in which new upgrade versions must always be deployed.

2. Description of the Prior Art

The present invention thus is in the field of medical technology and the information technology controller of the appertaining apparatuses.

In today's modern clinical units medical technology apparatuses are normally controlled by a computer, known as a control computer. For example, in the field of radiology a control computer is used to control MR systems, CT apparatuses or ultrasound apparatuses. The control computer serves to control the entire system and thus serves to control all participating components that, for example, can pertain to a gradient amplifier, an RF transmission amplifier, RF receiver devices, control modules to position the movable table, control panels at the apparatus, etc.

A control computer is normally a component of a control cabinet (console) and is connected with the hardware of the MR system via corresponding (normally proprietary) interfaces. Since these are normally proprietary interfaces and proprietary hardware modules, a control computer is normally designed in a highly customized manner for a specific type of MR system. A control computer thus normally controls only exactly one specific MR system, or one type of MR system.

A control computer normally comprises hardware-based and software-based modules. In the course of a continuous development, there are also new versions of control modules that are continuously to be considered. If a new generation of control modules should be offered, applied in an MR system, it has previously been necessary to exchange all platform-dependent portions of the control computer so that the new generation of the control computer (with the new control modules) can also be used for the respective MR system.

A significant disadvantage of the previous systems is only very few components of the previous MR system could be reused (for example only the magnet of the MR system). This would lead to the situation that implementation of new versions of control modules has often been declined since this would entail a cost expenditure that is too high.

In order to be able to use newer versions of control modules in spite of this problem, in the prior art it has been managed to port modules of the newer control version back to the systems of the older generations. However, the backporting to old platforms is very time-consuming and often corresponds (in terms of cost) to a new implementation since special limit conditions of the original control modules (for example computing power, data transfer capacities, memory capacities, operating systems etc.) had to be accounted for precisely. Moreover, such backporting was frequently not possible at all, or only to a limited extent, because the computing power of the control computer of the old generation is not sufficient for the control methods of the newer generation.

### SUMMARY OF THE INVENTION

An object of the present invention is to provide a method and a controller with which new versions of control modules for MR systems or other imaging systems can be implemented quickly and simply so that optimally many of the previous (MR) systems—including their control computers—can be retained.

In the following the solution to the object is described in relation to the claimed method. Features, alternative embodiments and/or advantages that are hereby mentioned are likewise applicable to the system and the computer program product and vice versa. The corresponding functional features of the method correspond to the physical modules, in particular the hardware modules, and the functionality of the hardware modules corresponds to the functionality of the method-related modules and vice versa.

A central aspect of the present invention is that platform-dependent and platform-independent control modules (also abbreviated as “modules” in the following) are differentiated in the controller. According to the invention, in the control method and/or control computer, portions are identified that are platform-dependent, thus dependent on a platform of the control computer and/or the MR system. Examples of such platform-dependent control modules can be, among other things, modules that are linked to the operating system or to the hardware of the respective computer. In addition to the class of platform-dependent control modules, there is a class or group of control modules that are in principle independent of a platform of the MR system and of the control computer. These are designated as platform-independent.

In the following the concepts that are used within the scope of this invention are explained in detail.

As noted above, the invention includes a controller of a medical imaging apparatus. The preferred embodiment of the invention concerns a controller for MR systems. For those skilled in the art it is apparent that all other medical technology apparatuses (and in particular imaging apparatuses) or other technical apparatuses that have a platform-dependent portion and a platform-independent portion can also be controlled with the solution according to the invention. The principle according to the invention is thus also applicable in, among other things, radiation therapy systems, CT (computed tomography) systems, PET (positron emission tomography) systems and US (ultrasound) systems. When an MR apparatus is discussed within the scope of this application, this is understood only as an example, such that all other types of imaging apparatuses are included in principle.

As used herein, “control method” means a hardware-based and/or software-based, computer-implemented control concept for the MR apparatus. The control method serves to activate all components of the MR system, for example the gradient system, the RF system, the positionable patient bed, interfaces, modules for data acquisition and data relaying, etc.

The “control computer” is an independent computer that can also be distributed to multiple computers within the framework of a client-server architecture and that is connected with the MR system via a proprietary interface (for example optical waveguide for data transfer according to a proprietary protocol) or CAN (controller area network) bus or other bus systems.

In the prior art an exact association between control computer and MR system was a mandatory requirement. A specific type of control computer thus matched exactly to a specific type of MR system. According to the invention, this association mechanism can advantageously become markedly more flexible.

According to the invention, in the control computer two different control segments or classes of control modules are differentiated: a physical segment and a logical segment. The logical segment of the control computer includes all control modules that are platform-independent, thus independent of an operating system or a hardware of the MR system (or of the control computer). In contrast to this, the physical segment includes all of those control modules that are platform-dependent and thus bound to hardware and/or to the operating system. The platform-dependent control modules are thus specifically fashioned for a specific type of MR system.

According to the invention, in addition to the (previous, old) control computer an additional control computer (designated in the following as an “auxiliary control computer”) is used. All control modules that have been identified (typically in advance) as associated with the logical segment of the control computer are swapped out to the auxiliary control computer. The auxiliary control computer is advantageously connected via a standardized interface (for example Ethernet, HTTP or the like) with the (previous) control computer, in particular with the physical segment of the control computer.

The (old) control computer is modified insofar as a data transfer module is prepared on the control computer. The data transfer module is designed to receive data of the auxiliary control computer and to prepare them for forwarding to the MR system.

Since the auxiliary control computer advantageously has standardized interfaces (both input interfaces and output interfaces), it is easily possible to implement upgrades or new versions of control modules on the auxiliary control computer without accompanying modifications to additional apparatuses (control computer or MR system) being necessary.

A primary feature of the present invention is therefore that the (previous) control computer is retained with regard to its hardware portions and this is henceforth to be operated as a “data pass-through station” for the MR system, as an adapter so to speak. This has the advantage that upgrades of control modules can be implemented without additional modification having to be made to the previous control computer or to the MR system or to the proprietary hardware. The logical portion or the logical segment of the original control computer is thereby displaced to the auxiliary control computer. The auxiliary control computer is thereby involved in a data exchange with the (old) control computer via an advantageously standardized interface (for example a network connection).

According to one aspect of the present invention, the control method activates the MR apparatus via at least one driver, wherein the driver is implemented as a software module on the control computer. In particular, the driver can be designed for only one specific component of the MR apparatus and/or for only one specific operating system.

According to an embodiment of the invention, the control modules that are implemented on the auxiliary control computer generate control signals that are relayed via an interface to the (previous) control computer. This receives these control signals via the data transfer module and prepares them for direct activation of the MR apparatus. It is also possible for the control computer to use the received control signals for indirect control of the MR system, for example via the use of a driver.

Different versions of the control method (for example upgrades) can be implemented in a simple manner via the division of the control method into a logical segment and a physical segment.

According to a preferred embodiment and depending on the application case, it is possible to connect an additional powerful computer to the auxiliary control computer in order to be able to control complex image processing tasks, for example. This could in particular ensue within the framework of a distributed computer system.

The term “platform” as used within the scope of the present invention includes primary aspects of the hardware and of the respective operating system. However, interfaces or the architecture of the apparatus and/or of the computer can also be encompassed by this term. All modules that are specifically related to a specific aspect of the platform are platform-dependent. For example, control modules are specifically described for a 16-bit system for Microsoft Windows or for a 32-bit system, such that they cannot be used for a UNIX/Linux system. In contrast to this, the term “platform-independent” means that the respective control modules are not limited to a specific platform or to a specific aspect of a platform. For example, a control module is platform-independent in the event that it controls when which RF pulse should be sent with which duration and amplitude in which shape. The activation of the gradient amplifier can likewise be implemented so as to be basically independent of a respective platform.

The invention concerns a control method for an MR apparatus as well as an extension of an existing control method. In both cases an auxiliary control computer is connected to the previous control computer. The auxiliary control computer advantageously has standardized interfaces and in particular has an interface to a console computer via which the control method is controlled and an (advantageously standardized) interface to the (previous) control computer. All the control modules that are independent of a platform are swapped out to the auxiliary control computer. These (platform-independent) control modules are also designated as a logical segment of the control computer within the scope of the invention. The physical segment, which combines or should identify the platform-dependent control modules on the (previous) control computer, is another example in addition to the logical segment.

In the extension of the previous control method the two aforementioned segments (the physical segment and the logical segment) of the (previous) control computer are initially to be identified. The logical segment is thereupon swapped out to the auxiliary control computer while the physical segment remains on the (previous) control computer. The previous control computer is adapted insofar as it now acts as a data pass-through station or, respectively, relay station. It is in particular supplemented with a data transfer module that receives control data of the auxiliary control computer and prepares it for relaying to the MR apparatus for control purposes. The (previous) control computer is thereby normally connected with the MR apparatus via a proprietary interface.

According to the invention the preparation and implementation of an MR measurement is executed on the auxiliary control computer and the commands are passed off to the (previous) control computer which relays these commands to the hardware of the MR apparatus with which it is connected. The relaying can be a direct relaying of the control signals to the MR apparatus. In a further case it can be necessary that the control signals must still be prepared so that they can be received by the MR system.

Since only the logical portion of the control method must run on the auxiliary control computer, the logical segment (with the respective control functionality) can also be displaced to computers of the MR system that are present anyway. For example, here the computer for image reconstruction or the system console can be used. In alternative embodiments it is also possible to distribute only portions of the logical segment to different auxiliary control computers that are present anyway or can also be newly provided.

An advantage is that the auxiliary control computer in principle does not need to be compatible with the previously present hardware and also does not need to be compatible with the existing MR system. A majority of the hardware that is already present (and that is very expensive in part and therefore can only be exchanged at a high cost) can therefore continue to be used. According to the invention this is achieved by the previous control computer continuing to be used exclusively to control the proprietary hardware (or more generally the proprietary platform) while its other tasks (for example complicated calculations to prepare and implement an MR measurement) can now be executed on the (normally more powerful) auxiliary control computer. So that there exists no bottleneck (in the information technology sense) between the previous control computer and the auxiliary control computer, it is provided according to the invention to provide an optimally standardized data connection with sufficient capacity between the control computer and the auxiliary control computer. The auxiliary control computer is typically a new computer generation that is generally equipped with improved capacity, power and resources and thus also comprises the logical functionalities of the old generation of the platform that is to be controlled.

The approach according to the invention can be broken up into two time phases. A first phase is a preparation phase and serves for the identification of the logical and physical segment on the (previous) control computer. In this preparation phase the control modules are, so to speak, first distributed once to the different control computers or, respectively, assigned to them. Network utilization criteria can hereby also be applied. In a second phase—the control phase—the actual activation of the MR system takes place. Here the control signals of the control modules are generated and prepared for relaying to the MR system. However, the control phase also includes the actual control of the MR measurement process. The preparation phase (as the name already suggests) is typically executed to prepare the actual measurement and thus is already executed beforehand. However, it is also possible that the two phases exhibit overlapping areas.

In the preceding the preparation of one auxiliary control computer was primarily discussed. However, it is likewise possible to provide multiple different auxiliary control computers here at which different control modules of the logical segment are implemented.

Via the architecture according to the invention with a control computer and an auxiliary control computer it is possible to deploy and implement different versions of control methods easily and without additional changes to the hardware of the MR apparatus or, respectively, to the existing control computer since the auxiliary control computer possesses an advantageously standardized interface and only pertains to the platform-independent portions.

A further object solution concerns a system to control an MR system with a control computer, an auxiliary control computer and a console computer. In a less complex, alternative embodiment of the invention, the functionality of the console computer is swapped out to another (existing) computer, for example to the previous control computer or to the auxiliary control computer.

The method can also be stored as a computer program product on a storage medium that can be imported into a computer via an interface.

As mentioned above, the interface between the MR system and the (previous) control computer is typically a proprietary interface. However, this term should also include those interfaces that are implemented with standard components and are merely based on a private (or proprietary) protocol.

### BRIEF DESCRIPTION OF THE DRAWINGS

FIG. 1 is a schematic representation of a control system according to the invention according to a preferred embodiment and

FIG. 2 is a workflow diagram of the control method according to the invention according to a preferred embodiment of the invention.

### DESCRIPTION OF THE PREFERRED EMBODIMENTS

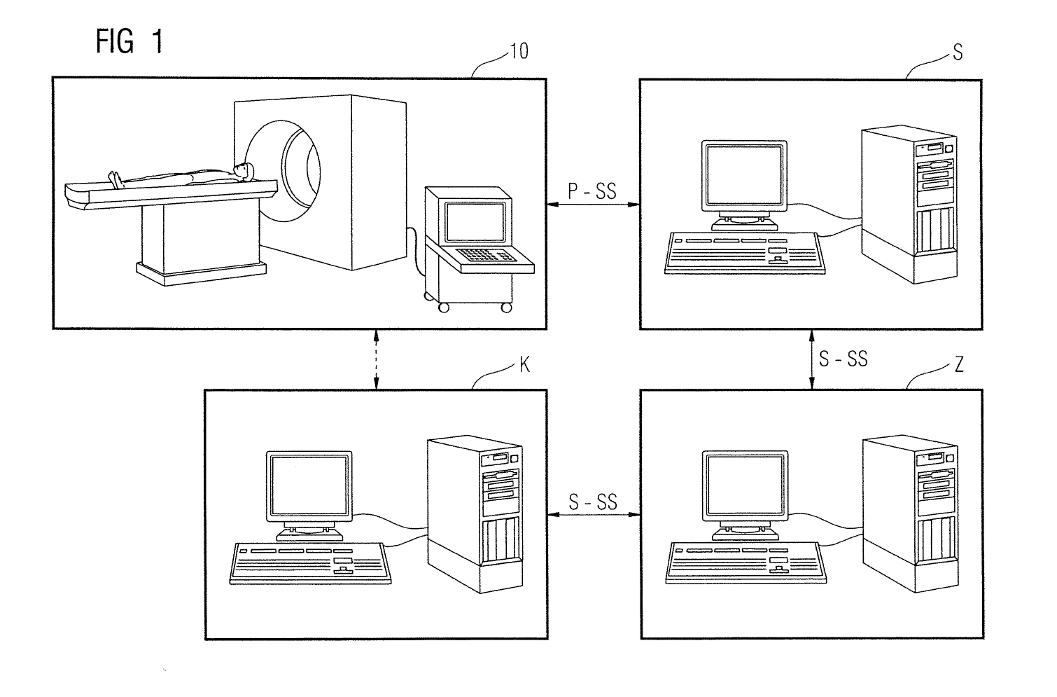

In the following the fundamental design of the control system is explained in detail in connection with FIG. 1.

An MR system 10 (that is also called an MR apparatus and can also comprise additional modules, for example computer-assisted control units) is connected to a control computer S via a proprietary interface P-SS. The control computer serves to control the MR system 10.

Furthermore, an auxiliary control computer Z is provided that communicates with the (previous) control computer S via an advantageously standardized interface S-SS.

Moreover, a console computer K is provided that can optionally be connected with the MR system 10 via a (direct, normally and advantageously likewise standardized) interface. The console computer K is advantageously and normally connected with the auxiliary control computer Z via a standard interface. A user can control the control method and, if necessary, input parameters via a provided user interface via the console computer K. The control computer S can also optionally be engaged in a data exchange with the console computer K via an—advantageously standardized—interface (not shown in FIG. 1) (for example to start up the control computer S).

In addition to the MR system 10, previous control methods according to the prior art comprise the control computer S and the console computer K.

According to the invention, the previous control system is expanded in that an auxiliary control computer Z is provided on which all platform-independent control modules of the control method are implemented. Since the auxiliary control computer Z advantageously possesses standardized interfaces (and in particular advantageously even has standardized interfaces exclusively), it is easily possible to execute new versions, updates/upgrades and/or additional changes to aspects of the control method.

The control method has a static portion and a dynamic portion. “Dynamic” in this context means that here aspects of the control method are encompassed that are relevant during the implementation of an MR measurement. All other aspects of the control method are classified as static. Static can also mean that the table (for example) should be driven to a determined position. The positioning of the table is in principle independent of the implementation of the MR measurement and must or, respectively, can normally be executed in advance. The control modules that are relevant to the positioning of the table can therefore be associated with the static portion. In principle the dynamic portion of the control modules is to be implemented in a significantly more complicated manner since this must be continuously reset [readjusted] in the implementation of an MR measurement. For example, the activation of the gradient system, the activation of the RF pulses at specific points in time with specific parameters etc. count among these.

As shown in FIG. 1, the MR system 10 is connected with the control computer S via a proprietary interface P-SS. The control computer S is typically connected with the auxiliary control computer Z via a standardized interface S-SS, which auxiliary control computer Z is in turn connected with the console computer K via a standardized interface S-SS. In FIG. 1 an interface (indicated with a dotted line) is likewise provide between the MR system 10 and the console computer K. However, this interface is optional and not absolutely necessary (and therefore is shown with a dotted line). For example, here control data of the user can be transferred directly to the MR system 10 or, respectively, parameters of the MR system 10 (for example the detection of physiological signals) can likewise be relayed to the console computer K for presentation to the user. In other embodiments it is possible to also provide standardized interfaces S-SS instead of the proprietary interfaces P-SS and vice versa. The proprietary interfaces can also be implemented via standardized components and be based on proprietary or standardized protocols.

Since an MR system 10 is normally a very complex system that normally consists of multiple modules, its activation is also a complex task. Therefore a specific association of control modules with MR components is typically provided. Specific control modules are thus typically precisely fashioned for specific components of the MR system 10 that normally are not compatible with one another. For example, a control module of a control method that is designed for a specific type of MR systems 10 (for example for a platform line B) is thus incompatible with control modules that are fashioned for another system type (for example a platform of line A or C). The incompatibility here is primarily based on the different hardware connections although the fundamental properties of the MR system 10 coincide in all types of MR systems (for example, all types or lines of MR systems cited in the preceding have three field gradients, RF transmitters and RF receivers etc.). However, the technical realization differs (for example in the use of different internal bus systems).

Accordingly, a console computer K is also typically set up for a specific type of MR system 10 so that here the console computer K can also not be exchanged without further changes. The console computers K are typically connected to the previous control computer S via a standard interface (Ethernet, for example). Control modules that serve for the activation of the proprietary hardware of the respective MR apparatus 10 via what are known as drivers wear and are implemented on the control computers S. Moreover, a control computer S also comprises logic or functionalities to implement an MR measurement. Depending on the control method, the control computers S then send specific control signals to the MR system 10 via the interface P-SS (for example gradient signals, RF signals and/or additional control signals).

The drivers that are implemented on the control computer S are linked to a concrete control computer S with regard to a proprietary hardware and are also created for an entire specific system. For this reason a control computer S in the prior art cannot be exchanged without significant redevelopment costs (and thus without the provision of an auxiliary control computer Z according to the present invention). In principle the platform-bound portions of the control method must be traced in all changes, which entails an immense development cost. According to the invention this is no longer necessary since all platform-independent portions of the control method are combined in the logical segment and are swapped out to the auxiliary control computer Z. The logical segment with the platform-independent control modules can thus be changed easily, such that here new versions and/or upgrades can be implemented without additional modifications.

The method can also be used recursively for every new generation of control computers S or auxiliary control computers Z. In other words, a bisection with regard to logical segment and physical segment can also be identified at an auxiliary control computer Z.

A significant advantage of the solution according to the invention is apparent in that an old hardware or, respectively, an old platform can also be controlled with a new auxiliary control computer Z. Only the previous drivers thus remain on the old system (thus on the old control computer S). Moreover, a new part—in particular a data transfer module—is to be provided which receives or, respectively, imports the control data from the logical segment of the auxiliary control computer Z and passes it through to the driver of the control computer S (and thus indirectly to the MR system 10).

An additional advantage is apparent in that multiple different logical segments (possibly with regard to different platforms) can also be bundled or, respectively, communalized on a common auxiliary control computer Z. Costs and development expenditure can therefore be markedly reduced since changes to the control method or to modules of the previous control method can be executed without hardware modifications.

The changes with regard to the control method thereby pertain not only to the concrete control data; rather, it is likewise possible to provide a different user interface or to vary the manner of data access. This is possible in that all platform-independent portions are identified and processed separately. Only the hardware-bound and operating system-bound portions remain on the previous control computer S, which is concretely fashioned with regard to an MR system 10 (or to a specific type of MR system 10).

According to the invention, control modules can thus also be exchanged that relate to different lines of MR apparatuses 10 (for example for platform lines A, B or C cited in the preceding). The platforms do not need to be identical in terms of or specific to the manufacturer, such that control modules from different manufacturers (of MR apparatuses 10 and/or of associated control computers) can also be exchanged. The flexibility in clinical practice can therefore be markedly increased.

In principle it is possible to provide a new computer Z as a control computer on which the logical portions of the control method can be implemented. However, in an alternative embodiment it is also possible to not provide a separate auxiliary control computer Z here but rather to distribute the logical segment to already existing computers, for example to an image reconstruction computer. Control computer S and auxiliary control computer Z should advantageously have a real-time operating system.

According to a preferred embodiment, the physical segment predominantly comprises all those control modules that are dependent on a platform (in particular on the hardware and on the operating system) of the MR apparatus 10. According to an advantageous development of the invention, however, the control modules of the physical segment are also dependent on the platform (in particular on the hardware and on the operating system) of the respective control computer S. The logical segment results from the complementary set for the physical segment and, depending on the embodiment, can comprise different control modules.

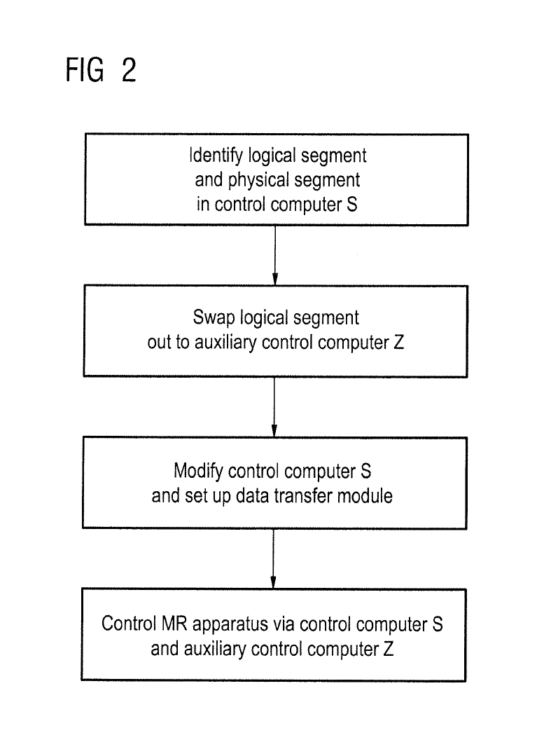

In the following the basic workflow of a method according to the invention is explained in detail according to a preferred embodiment of the invention in connection with FIG. 2.

In a first step the logical segment and physical segment are identified on the previous control computer S. The logical segment comprises all control modules that are independent of a platform of the control computer S and/or the MR system 10. The platform basically refers to the present hardware and the operating system. The physical segment comprises all remaining control modules, thus in particular those that are dependent on the platform.

In a second step the logical segment with its control modules is swapped out to the auxiliary control computer Z. In a further intermediate step (not shown in FIG. 2), the respective interfaces between the computers are modified. In particular, only the previous control computer S communicates with the MR system 10 via a proprietary interface or via a proprietary protocol while all other instances are engaged in data exchange via advantageously standardized interfaces.

In a further step, the previous control computer S is adapted in that an additional data transfer module is set up. The data transfer module serves to receive control signals that are generated and/or sent by the auxiliary control computer Z. These control signals are then processed further and relayed to the MR apparatus to control it. For example, conversion and formatting functionalities are to be cited here.

The method steps mentioned in the preceding can be associated with a preparation phase. In the event that it is technically possible, the method steps can also be executed in a different order.

In a last step the MR apparatus 10 is actually controlled. The control ensues via the control computer S and the auxiliary control computer Z. The last step is thus executed in a control phase that is in principle independent of the preparation phase.

Although modifications and changes may be suggested by those skilled in the art, it is the intention of the inventor to embody within the patent warranted hereon all changes and modifications as reasonably and properly come within the scope of his contribution to the art.

I claim as my invention:

1. A control method for operating a medical imaging apparatus comprising the steps of:
using a control platform to execute a control procedure to control a medical imaging apparatus via a plurality of control modules;
among said plurality of control modules, designating platform-dependent control modules that are implemented by a control computer of the medical imaging apparatus and designating platform-independent control modules that are implemented by an auxiliary control computer of the medical imaging apparatus;
placing the auxiliary control computer in data exchange communication with the control computer via a standardized interface; and
in said control computer, providing a data transfer module and, via said data transfer module, receiving control data from the auxiliary control computer via said standardized interface and relaying said control data from the auxiliary control computer to the medical imaging apparatus via the control computer for, at least in part, controlling operation of said medical imaging apparatus dependent on the control data from the auxiliary control computer.

2. A method as claimed in claim 1 comprising controlling said medical imaging apparatus with said platform via at least one driver that is implemented at the control computer and interfacing said driver with said control computer via a driver platform.

3. A method as claimed in claim 2 wherein said medical imaging apparatus comprises a hardware component and an imaging component and comprising operating at least one of said hardware component and said imaging apparatus via said driver.

4. A method as claimed in claim 2 comprising embodying said driver in an operating system of the medical imaging apparatus.

5. A method as claimed in claim 1 comprising loading a modified version of said platform into the auxiliary control computer for use in the auxiliary control computer with no physical modifications to said control computer.

6. A method as claimed in claim 1 comprising configuring a plurality of different versions of said control platform for use with different console computers having different platforms and being configured for operating different types of imaging apparatuses.

7. A method as claimed in claim 1 comprising operating said control procedure through a console computer, and placing said console computer in a further data exchange communication with said auxiliary control computer via a further standardized interface.

8. A method as claimed in claim 7 comprising also placing said console computer in data exchange communication with said control computer.

9. A method as claimed in claim 7 comprising also placing said control computer in direct data exchange communication with said medical imaging apparatus.

10. A method as claimed in claim 1 comprising operating said control computer and said auxiliary control computer with a real-time operating system.

11. A method as claimed in claim 1 wherein said imaging apparatus comprises hardware components that are exclusively controllable by respective proprietary interfaces, and comprising configuring said control computer to control said hardware components via said proprietary interfaces.

12. A method as claimed in claim 1 comprising re-designating at least one of said platform-independent control modules for operation thereof by said auxiliary control computer.

13. A method as claimed in claim 1 comprising employing, as said control modules, control modules selected from the group consisting of static control modules and dynamic control modules.

14. A method as claimed in claim 1 comprising executing said control procedure in a preparation phase and a control phase and, in said preparation phase, identifying logical and physical segments of said control computer and, in said control phase, controlling imaging by said medical imaging apparatus via said control computer and said auxiliary control computer.

15. A method as claimed in claim 1 wherein said medical imaging system is a magnetic resonance system and comprising, from said control computer and said auxiliary control computer, implementing said control procedure to control at least one of a radio-frequency transmission amplifier, a radio-frequency receiver, and a movable patient table of said magnetic resonance system.

16. A method as claimed in claim 15 wherein each of radio amplifier, said radio-frequency transmission amplifier, said radio-frequency receiver, and said movable patient table comprises a respective proprietary interface, and comprising controlling said at least one of said gradient amplifier, said radio-frequency transmission amplifier, said radio-frequency receiver and said movable patient table via the respective primary interface thereof.

17. A method as claimed in claim 16 comprising forming said respective proprietary interfaces to includes respective proprietary cables.

18. A method as claimed in claim 1 comprising forming said platform from components selected from the group consisting of hardware of said control computer, hardware of said imaging apparatus, interface cables, an operating system of said control computer, an operating system of said imaging apparatus, a computer architecture of the control computer and the imaging apparatus, and drivers.

19. A method to extend a control system of a medical imaging apparatus that uses a control platform to control a medical imaging apparatus via a plurality of control modules, comprising the steps of:
placing an auxiliary control computer in data exchange communication with a control computer via a standardized interface;
designating a logic segment in said control computer comprising platform-independent control modules that are then implemented by the auxiliary control computer; and
modifying a physical segment of said control computer comprising platform-dependent control modules and receiving control data from the auxiliary control computer with the modified segment via said standardized interface and relaying said control data from the auxiliary control computer to the medical imaging apparatus for, at least in part, controlling operation of said medical imaging apparatus dependent on the control data from the auxiliary control computer.

20. A control system for a medical imaging apparatus comprising:
a control platform that controls a medical imaging apparatus via a plurality of control modules;
a control computer;
an auxiliary computer;
said plurality of control modules comprising platform-dependent control modules that are implemented by the control computer of the medical imaging apparatus and designating platform-independent control modules that are implemented by the auxiliary control computer;
a standardized interface that places the auxiliary control computer in data exchange communication with the control computer; and
said control computer comprising a data transfer module that receives control data from the auxiliary control computer via said standardized interface and relays said control data from the auxiliary control computer to the medical imaging apparatus via the control computer for, at least in part, controlling operation of said medical imaging apparatus dependent on the control data from the auxiliary control computer.

21. A non-transitory computer-readable storage medium encoded with programming instructions, said storage medium being loaded into a control system of a medical imaging apparatus comprising a control computer and an auxiliary control computer, and said programming instructions causing said control computer and said auxiliary control computer to:
use a control platform to control a medical imaging apparatus via a plurality of control modules;
among said plurality of control modules, designate platform-dependent control modules that are implemented by a control computer of the medical imaging apparatus and designating platform-independent control modules that are implemented by an auxiliary control computer of the medical imaging apparatus;
place the auxiliary control computer in data exchange communication with the control computer via a standardized interface; and
in said control computer, provide a data transfer module and, via said data transfer module, receiving control data from the auxiliary control computer via said standardized interface and relaying said control data from the auxiliary control computer to the medical imaging apparatus via the control computer for, at least in part, controlling operation of said medical imaging apparatus dependent on the control data from the auxiliary control computer.
